(19) United States
(12) Patent Application Publication
(10) Pub. No.: US 2018/0140276 A1
(43) Pub. Date: May 24, 2018

(54) Wireless Probe Type Ultrasound Scanner Machine

(71) Applicant: Sonostar Technologies Co., Limited, Guangzhou, Guangdong (CN)

(72) Inventors: Weizhong Cai, Guangzhou, Guangdong (CN); Jianfa Wang, Guangzhou, Guangdong (CN); Chengming Liang, Guangzhou, Guangdong (CN)

(21) Appl. No.: 15/568,544

(22) PCT Filed: Aug. 23, 2016
(86) PCT No.: PCT/CN2016/096445

§ 371 Date: Oct. 23, 2017

(30) Foreign Application Priority Data
Aug. 24, 2015 (CN) 201520635748.9

(51) Int. Cl. A61B 8/00

(52) U.S. Cl. CPC A61B 8/4472; A61B 2560/04; A61B 8/56; A61B 8/4455

(57) ABSTRACT

The present invention proposes a wireless probe type ultrasound scanner which comprises a housing, a housing cover, a transducer, a mainboard, a battery compartment, a battery and a backplane; the housing is matched and connected with the housing cover; the mainboard, the battery compartment, the battery and the backplane are arranged in a cavity body formed by the housing and the housing cover; the mainboard and the transducer are welded together; the mainboard is communicated with the transducer; the mainboard, the battery compartment, the backplane and the battery are laminated in order, wherein the battery is clamped on the battery compartment, a setting hole arranged on the end part of the transducer in a way of penetrating through the housing cover is clamped with the housing cover, and a Bluetooth and a power switch are arranged on the mainboard.

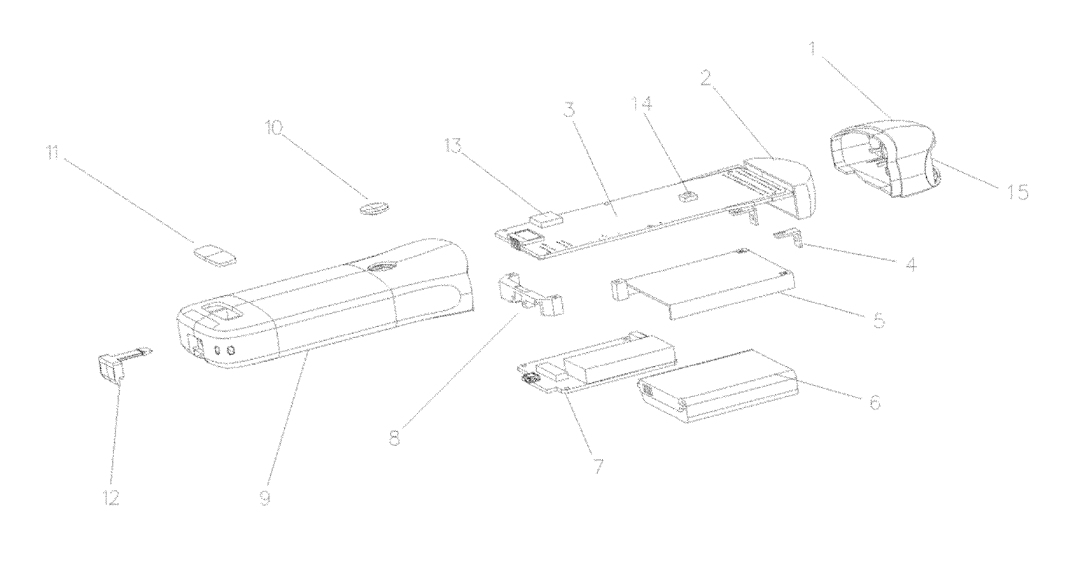

### CROSS REFERENCE OF RELATED APPLICATION

This is a U.S. National Stage under 35 U.S.C 371 of the International Application PCT/CN2016/096445, filed Aug. 23, 2016, which claims priority under 35 U.S.C. 119(a-d) to CN 201520635748.9, filed Aug. 24, 2015.

### BACKGROUND OF THE PRESENT INVENTION

### Field of Invention

The present invention relates to a small ultrasound diagnosis scanner, and more particularly to a wireless probe type ultrasound scanner.

### Description of Related Arts

A traditional ultrasound machine is often composed of a host and a probe, resulting in large volume and inconvenient carrying; the use of the probe and the host connected with solid lines is also inconvenient; however, a hand-held ultrasound scanner is relatively light and small, and is realized through wired connection of a separated type probe, an externally connected power supply and the host. The separated type structure is relatively simple and convenient in use, while line connection still brings inconvenience.

### SUMMARY OF THE PRESENT INVENTION

The present invention proposes a wireless probe type ultrasound scanner, which integrates a host into a probe, and has extremely simple structure, powerful function, convenient use and low environmental requirement.

To achieve the above object, the present invention provides a wireless probe type ultrasound scanner which comprises:

a housing, a housing cover, a transducer, a mainboard, a battery compartment, a battery and a backplane; wherein:

the housing is matched and connected with the housing cover, and the mainboard, the battery compartment, the battery and the backplane are arranged in a cavity body formed by the housing and the housing cover;

the mainboard and the transducer are welded together; the mainboard is communicated with the transducer; and the mainboard, the battery compartment, the backplane and the battery are laminated in order, wherein the battery is clamped on the battery compartment;

an end portion of the transducer passes through a setting hole provided in the housing cover and is clamped with the housing cover; and

a WiFi (wireless fidelity) and a power switch are arranged on the mainboard.

The wireless probe type ultrasound scanner further comprises multiple transducer fixing brackets; wherein: the transducer fixing brackets are connected between the battery compartment and the mainboard; and each of the transducer fixing brackets has an L shape.

The wireless probe type ultrasound scanner further comprises a circuit board fixing bracket; an end part of the circuit board fixing bracket penetrates through a plate fixing hole provided in the housing; the circuit board fixing bracket is clamped with the plate fixing hole; and the circuit board fixing bracket is electrically connected with the battery and the mainboard respectively.

The circuit board fixing bracket is provided with an externally connected power charging interface.

The circuit board fixing bracket is provided with a wireless charging device.

The wireless probe type ultrasound scanner further comprises a charging interface rubber plug, and the charging interface rubber plug is arranged on a power interface.

A light-transparent hood and a switch key are arranged on the housing; the switch key is arranged correspondingly to the power switch;

and the mainboard is provided with a job state indicator lamp correspondingly to the light-transparent hood.

Compared with the prior art, in the wireless probe type ultrasound scanner provided by the present invention, a host is integrated into a probe, and a circuit and a structure are extremely integrated. The wireless probe type ultrasound scanner has a built-in micro router to implement wifi connection communication with smart phones, tablet PCs and other intelligent terminals; the mobile phone and the tablet PCs display ultrasonic images and achieve powerful software function. The scanner is compact and has wireless connectivity, so it's convenient to use.

The wireless probe type ultrasound scanner provided by the present invention has simple structure and comprises an operating platform; the operating platform of the present invention is a platform based on visual carriers, such as mobile phones, PCs, and computers, having a wireless function, and is used with the present invention, wherein in the present invention, the host is integrated into the probe, so that the present invention has the superior characteristics of simple structure, convenient operation, low cost, low environment building requirement, etc.

### BRIEF DESCRIPTION OF THE DRAWINGS

To more clearly describe the technical solutions in the embodiments of the present invention or in the prior art, the drawings required to be used in the description of the embodiments or the prior art will be simply presented below. Apparently, the drawings in the following description are merely some embodiments of the present invention, and for those ordinary skilled in the art, other drawings can also be obtained according to the drawings without contributing creative labor.

The drawing is an exploded view of a wireless probe type ultrasound scanner provided by the present invention.

### DETAILED DESCRIPTION OF THE PREFERRED EMBODIMENT

The technical solutions in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

A wireless probe type ultrasound scanner provided by the present invention comprises a wireless signal receiver, i.e., a “terminal” for integrating a host into a probe. The wireless probe type ultrasound scanner only has the wireless signal receiver without transmitting capability, further comprises a device of receiving and transmitting hardware, and has a device for executing bidirectional communication of receiving and transmitting hardware on a bidirectional communication link. The device comprises: a honeycomb or other communication devices, i.e., a honeycomb or other communication devices with a single-line display or multi-line display or without multi-line display; a personal communications service (PCS) which can combine voice, data processing, fax and/or data communication capability; a personal digital assistant (PDA) which comprises a radio frequency receiver, a pager, an Internet/Intranet accessor and a network browser; and a conventional laptop and/or palm computer or other devices, i.e., a conventional laptop and/or palm computer or other devices having and/or comprising a radio frequency receiver. The “terminal” used herein is portable and transportable or is suitable and/or is configured for local operation, and/or is operated in other positions in a distribution form. The “terminal” used herein can also be communicated and networked, can be, for example, based on PDA and a mobile Internet device (MID) and/or have a mobile phone, and can also be connected with the devices of an intelligent television, etc. for use.

Those skilled in the art can understand that a remote terminal used herein can complete operation based on but not limited to an executable platform program of a cloud composed of a computer, a network host, a single network server, a set of a plurality of network servers or a plurality of servers. A connection network can realize communication through any communication mode, including but not limited to mobile communication based on 3GPP, LTE and WIMAX, computer network communication based on TCP/IP and UDP and a near field wireless transmission mode based on Bluetooth and infrared transmission standards.

As shown in the drawing, a wireless probe type ultrasound scanner comprises a housing 9, a housing cover 1, a transducer 2, a mainboard 3, a battery compartment 5, a battery 6 and a backplane 7; wherein: the housing 9 is matched and connected with the housing cover 1; the mainboard 3, the battery compartment 5, the battery 6 and the backplane 7 are arranged in a cavity body formed by the housing 9 and the housing cover 1; the mainboard 3 and the transducer 2 are welded together; the mainboard 3 is communicated with the transducer 2; the mainboard 3, the battery compartment 5, the backplane 7 and the battery 6 are laminated in order, wherein the battery 6 is clamped on the battery compartment 5, an end portion of the transducer 2 passes through a setting hole 15 provided in the housing cover 1 and is clamped with the housing cove 1, and a Bluetooth 13 and a power switch 14 are arranged on the mainboard 3.

The WIFI (wireless fidelity) involved in the present invention is essentially a wireless connection mode; other wireless connection modes such as Bluetooth shall be regarded within the protection scope of the present invention and fall into the protection scope of the present invention. The operating platform (software part) corresponding to the terminal is disclosed on a network, and can be searched and downloaded through the network, and will not be repeated again.

The wireless probe type ultrasound scanner further comprises transducer fixing brackets 4; the transducer fixing brackets 4 are connected between the battery compartment 5 and the mainboard 3; a plurality of transducer fixing brackets 4 are arranged; and each of the transducer fixing brackets 4 has an L shape.

The wireless probe type ultrasound scanner further comprises a circuit board fixing bracket 8; an end part of the circuit board fixing bracket 8 penetrates through a plate fixing hole provided in the housing 9; the circuit board fixing bracket 8 is clamped with the plate fixing hole; and the circuit board fixing bracket 8 is electrically connected with the battery 6 and the mainboard 3 respectively.

The circuit board fixing bracket 8 is provided with an externally connected power charging interface.

The circuit board fixing bracket 8 is provided with a wireless charging device.

The wireless probe type ultrasound scanner further comprises a charging interface rubber plug 12, and the charging interface rubber plug 12 is arranged on a power interface.

A light-transparent hood 11 and a switch key 10 are arranged on the housing; the switch key 10 is arranged correspondingly to the power switch 14, wherein after the power switch key finishes starting, the power switch key is switched into an image freezing/unfreezing function key; the light-transparent hood 11 is arranged correspondingly to the WIFI and used for displaying electric quantity and a WIFI signal state; and the mainboard is provided with a job state indicator lamp correspondingly to the light-transparent hood 11.

The above is just preferred embodiments of the present invention and is not intended to limit the present invention. Any modification, equivalent replacement, improvement, etc. made within the spirit and the principle of the present invention shall be contained within the protection scope of the present invention.

1. A wireless probe type ultrasound scanner, wherein a host is integrated with a probe to form a whole in the wireless probe type ultrasound scanner which comprises:
a housing, a housing cover, a transducer, a mainboard, a battery compartment, a battery and a backplane; wherein:
the housing is matched and connected with the housing cover, and the mainboard, the battery compartment, the battery and the backplane are arranged in a cavity body formed by the housing and the housing cover;
the mainboard and the transducer are welded together; the mainboard is communicated with the transducer; and the mainboard, the battery compartment, the backplane and the battery are laminated in order, wherein the battery is clamped on the battery compartment;
an end portion of the transducer passes through a setting hole provided in the housing cover and is clamped with the housing cover; and
a WiFi (wireless fidelity) and a power switch are arranged on the mainboard.

2. The wireless probe type ultrasound scanner according to claim 1, wherein:
the wireless probe type ultrasound scanner further comprises multiple transducer fixing brackets; the transducer fixing brackets are connected between the battery compartment and the mainboard; and each of the transducer fixing brackets has an L shape.

3. The wireless probe type ultrasound scanner according to claim 1, wherein:
the wireless probe type ultrasound scanner further comprises a circuit board fixing bracket; an end part of the circuit board fixing bracket penetrates through a plate fixing hole provided in the housing; the circuit board fixing bracket is clamped with the plate fixing hole; and the circuit board fixing bracket is electrically connected with the battery and the mainboard respectively.

4. The wireless probe type ultrasound scanner according to claim 3, wherein:
the circuit board fixing bracket is provided with an externally connected power charging interface.

5. The wireless probe type ultrasound scanner according to claim 3, wherein:
the circuit board fixing bracket is provided with a wireless charging device.

6. The wireless probe type ultrasound scanner according to claim 3, wherein:
the wireless probe type ultrasound scanner further comprises a charging interface rubber plug, and the charging interface rubber plug is arranged on a power interface.

7. The wireless probe type ultrasound scanner according to claim 1, wherein:
a light-transparent hood and a switch key are arranged on the housing; the switch key is arranged correspondingly to the power switch; and
the mainboard is provided with a job state indicator lamp correspondingly to the light-transparent hood.
